(19) United States
(12) Patent Application Publication
(10) Pub. No.: US 2022/0168137 A1
(43) Pub. Date: Jun. 2, 2022

(54) HEATING APPARATUS FOR AN ORTHOTIC DEVICE

(71) Applicant: Costello Prosthetic Warmers LLC, Liverpool, NY (US)

(72) Inventor: Bryan Costello, Liverpool, NY (US)

(73) Assignee: Costello Prosthetic Warmers LLC, Liverpool, NY (US)

(21) Appl. No.: 17/675,267

(22) Filed: Feb. 18, 2022

Related U.S. Application Data
(63) Continuation-in-part of application No. 17/236,643, filed on Apr. 21, 2021.
(60) Provisional application No. 63/030,346, filed on May 27, 2020.

(51) Int. Cl. A61F 7/02; A61F 5/01

(52) U.S. Cl. CPC A61F 7/02; A61F 2007/0029; A61F 5/013; A61F 5/0123

(57) ABSTRACT

An orthotic device includes an orthotic appliance formed by at least two laminate layers and a heating apparatus including a heating element embedded between the at least two layers of the orthotic appliance. The heating apparatus further includes a power supply operatively coupled to the heating element. The heating element selectively heats the orthotic appliance. The heating element includes a wireless receiving coil. The power supply includes a wireless charging transceiver such that when power is supplied to the wireless charging transceiver, a current is induced within the wireless receiving coil to supply power to the heating element.

### CROSS-REFERENCE TO RELATED APPLICATIONS

This application is a continuation-in-part of U.S. patent application Ser. No. 17/236,643 filed Apr. 21, 2021, which claims the benefit of U.S. Patent Application No. 63/030,346, filed May 27, 2020, the contents of each of which are hereby incorporated by reference in their entireties.

### FIELD OF THE INVENTION

This invention relates generally to heating apparatuses and, more particularly, to a heating apparatus for an orthotic device whereby the orthotic device is configured to selectively impart heat to the area of orthotic support and treatment via the heating apparatus.

### BACKGROUND OF THE INVENTION

Orthoses are generally braces used to prevent or assist movement of a limb or the spine while orthotics are appliances which support, align, straighten or otherwise improve the functioning of a limb or other part of the body. For sake of clarity, the term orthotic device as used throughout the specification and claims shall mean either an orthosis or orthotic, and the term limb shall mean a limb or any other body part, unless context dictates otherwise. Custom orthotic devices are generally laminated appliances which are specifically designed for a particular individual. These custom devices may also include metal or plastic components, such as hinges for controlling movement, as well as padding to improve user comfort. Straps and/or other constraints may be used to removably secure the orthotic device on the user's limb.

In addition to using an orthotic device to provide support and assistance, application of heat is also frequently employed in patient treatment paradigms and to improve patient comfort. This heat is typically applied through use of a heating blanket, heating pad or similar device. Many commercially available products, however, are not designed to target specific areas, although there are some blankets that are generally shaped to roughly conform to the part of the body being heated, such as the shoulders, knees or elbows. Nonetheless, each of these devices cannot focus heating to a specific treatment area. Moreover, these devices are not readily amenable to use while the orthosis/orthotic device is being worn or used so as to apply heat directly to the underlying limb.

Therefore, it would be desirable to have a system and method of adjusting the temperature of an orthotic device when being worn so as to provide additional comfort to the wearer and to improve compliance with a physician's treatment orders. The present invention satisfies these, as well as other, needs.

### BRIEF SUMMARY OF THE INVENTION

In accordance with an aspect of the present invention, one exemplary embodiment may be directed towards an orthotic device comprising an orthotic appliance formed by at least two laminate layers and a heating apparatus. The at least two layers includes a first laminate layer and a second laminate layer. The heating apparatus includes a heating element embedded between the first laminate layer and the second laminate layer. The heating apparatus may further include a power supply operatively coupled to the heating element, wherein the heating element selectively heats the orthotic appliance. The heating element may comprise a flexible heat strip and the power supply may be a battery. The battery may be configured to be worn on or proximate to the orthotic appliance and may be coupled to the battery via a wired connection. At least one wire may pass through an outer surface of the orthotic appliance whereby the at least one wire is connected between the power supply and the heating element.

In accordance with a further aspect of the present invention, the heating element may include a wireless receiving coil, wherein the power supply includes a wireless charging transceiver, and wherein when power is supplied to the wireless charging transceiver, a current is induced within the wireless receiving coil to supply power to the heating element.

In accordance with another aspect, the temperature output of the heating element may be selectively adjustable and the heating element may have a maximum temperature output selected such that a surface temperature of the orthotic appliance does not exceed about 80 degrees Celsius.

In accordance with another aspect, the present invention may be directed to a method of selectively heating an orthotic device being worn on at least a portion of a body. The method may comprise: a) placing the orthotic device on the at least a portion of the body, wherein the orthotic device comprises an orthotic appliance formed by at least two laminate layers and a heating apparatus, wherein the at least two laminate layers include a first laminate layer and a second laminate layer, and wherein the heating apparatus includes a heating element embedded between the first laminate layer and the second laminate layer; and b) selectively powering the heating element to output heat for heating the orthotic device.

Other objects and advantages of the present invention will become apparent from the following description taken in connection with the accompanying drawings, wherein is set forth by way of illustration and example, embodiments of this invention.

### BRIEF DESCRIPTION OF THE DRAWINGS

The accompanying drawings form a part of this specification and are to be read in conjunction therewith, wherein like reference numerals are employed to indicate like parts in the various views, and wherein:

FIG. 1 is a perspective view of an exemplary orthotic device including an embodiment of a heating apparatus in accordance with an aspect of the present invention;

FIG. 2 is a rear perspective view of the exemplary orthotic device shown in FIG. 1;

FIG. 3 is a perspective view of an exemplary embodiment of an elbow orthotic device including a heating apparatus in accordance with another aspect of the present invention;

FIG. 4 is a perspective view of an exemplary embodiment of a knee ankle foot orthotic device including a heating apparatus in accordance with a further aspect of the present invention;

FIG. 5 is a partial transverse cross section view of the embodiment of the orthotic device shown in FIGS. 1 and 2, taken generally along line 5-5 in FIG. 1;

FIG. 6 is a perspective view of an exemplary heating apparatus configured for use within the orthotic device shown in FIGS. 1 and 2;

FIG. 7 is a perspective view of an alternative exemplary orthotic device including a further embodiment of a heating apparatus in accordance with another aspect of the present invention;

FIG. 8 a partial cross section view of the alternative embodiment of the orthotic device shown in FIG. 7; and

FIG. 9 is a cross section view of an exemplary battery pack configured for use with the alternative embodiment of a heating apparatus shown in FIG. 7.

### DETAILED DESCRIPTION OF THE INVENTION

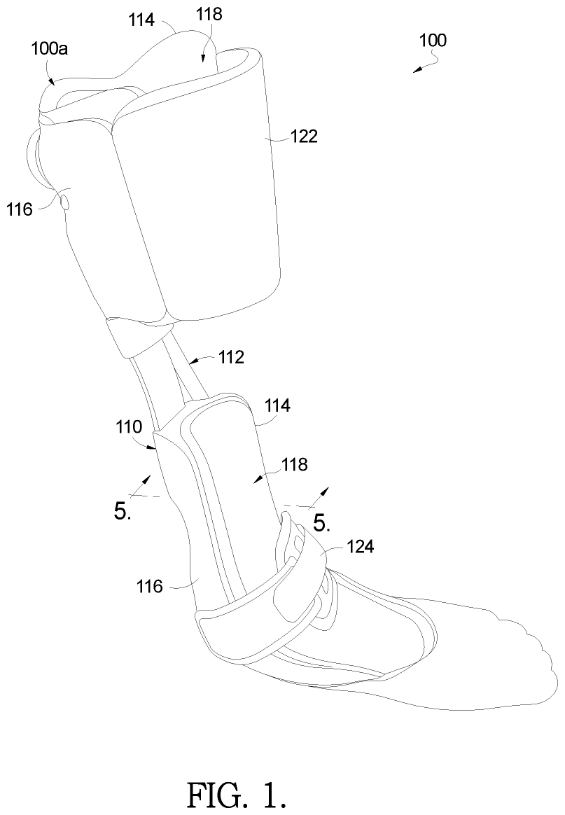

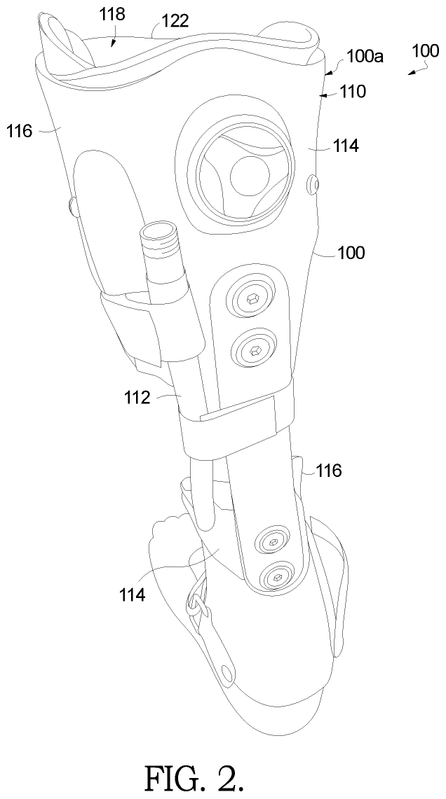

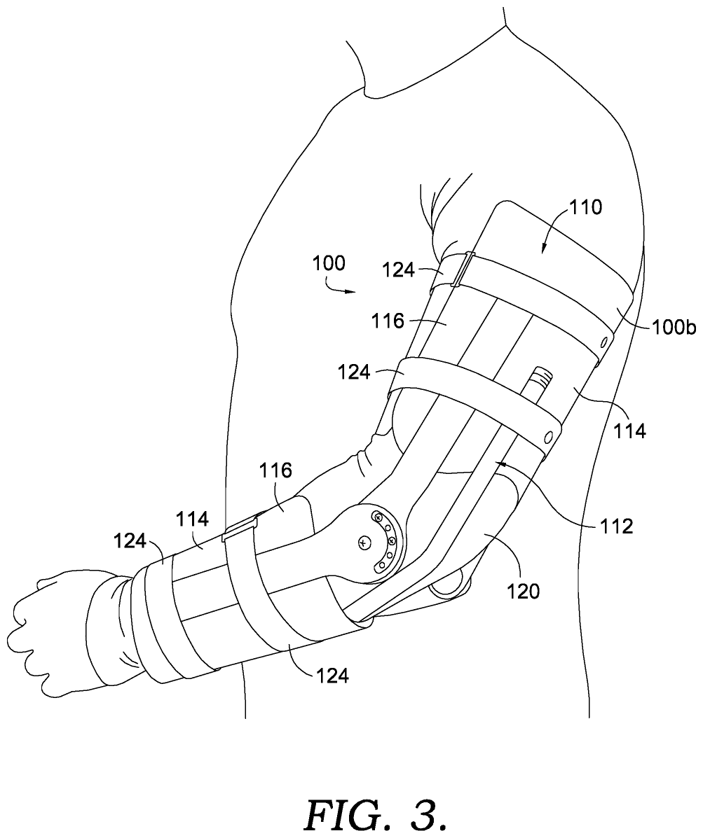

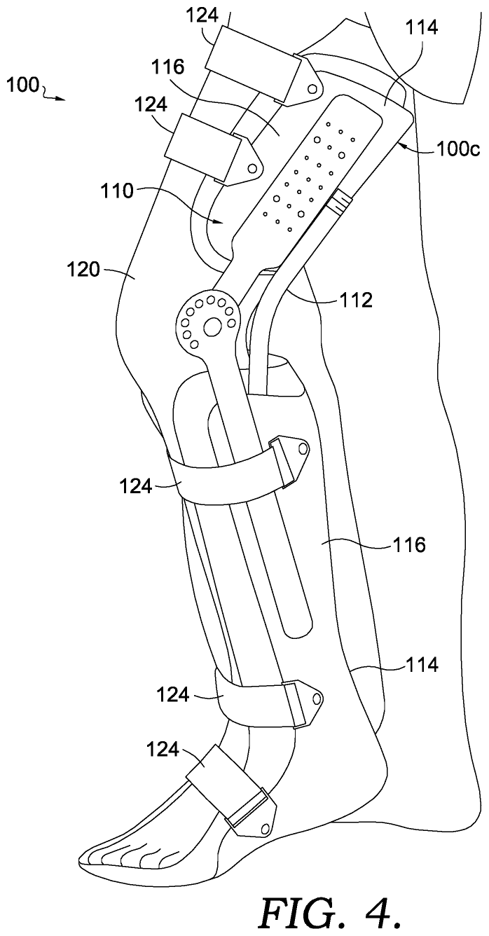

With reference to FIGS. 1-4 exemplary embodiments 100a, 100b, 100c of an orthotic device 100 are shown, with an ankle foot orthotic device (AFO) 100a (FIGS. 1 and 2), an elbow orthotic device (EO) 100b (FIG. 3) and a knee ankle foot orthotic device (KAFO) 100c (FIG. 4). Orthotic device 100 generally includes an orthotic appliance 110 and a heating apparatus 112, with the differences between orthotic devices 100a, 100b, 100c directed toward the structural shape of orthotic appliance 110 which is configured to receive the user's limb or other part of the body therein, and, in some cases, the specific layout of the various components of heating apparatus 112 depending upon the size and/or needs of orthotic appliance 110 or user preference. However, the structural fabrication of the orthotic device 100 and general construction of heating apparatus 112 are similar between orthotic devices 100a, 100b, 100c as will be discussed in greater detail below.

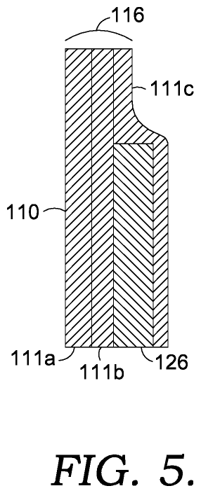

Orthotic appliance 110 generally includes one or more brace units 114 each having a generally curved sidewall 116 defining an interior area 118 (see FIGS. 1 and 2) that is configured to receive the user's limb (or other body part) 120 therein (see FIGS. 3 and 4). For sake of clarity, reference numeral 120 will be defined as a user's limb, but it should be understood that orthotic appliance 110 may be adapted for any body part, as needed, and is not limited solely to the limbs. Additional bracing, e.g. brace subunit 122 (see FIGS. 1 and 2), or adjustable strapping, such as VELCRO strap 124 (see FIGS. 1, 3 and 4), may be provided to secure user's limb 120 within brace unit 114 during use. In one aspect of the present invention, orthotic appliance 110 may be cast, such as through use of an epoxy resin. In another aspect and as shown in FIG. 5, orthotic appliance 110 may be manufactured as a laminate (such as layers 111a, 111b, 111c) using materials such as, but not limited to, carbon fiber, fiber glass or plastics. In either approach (casting or lamination), a heating element 126 (discussed in greater detail below) of heating apparatus 112 is embedded within side wall 116 of orthotic appliance 110. For example, hearing element 126 may be disposed between two of the laminate layers, such as layers 111b and 111c. Heating element 126 could also be disposed between layers 111a and 111b, for example. It should be noted that, while shown and described as having heating element 126 located on only one brace unit 114, each brace unit 114 may include a respective heating element 126 embedded therein, and each respective heating element 126 may be selectively powered to target application of heat to one or more desired brace units 114, as will be described in greater detail below.

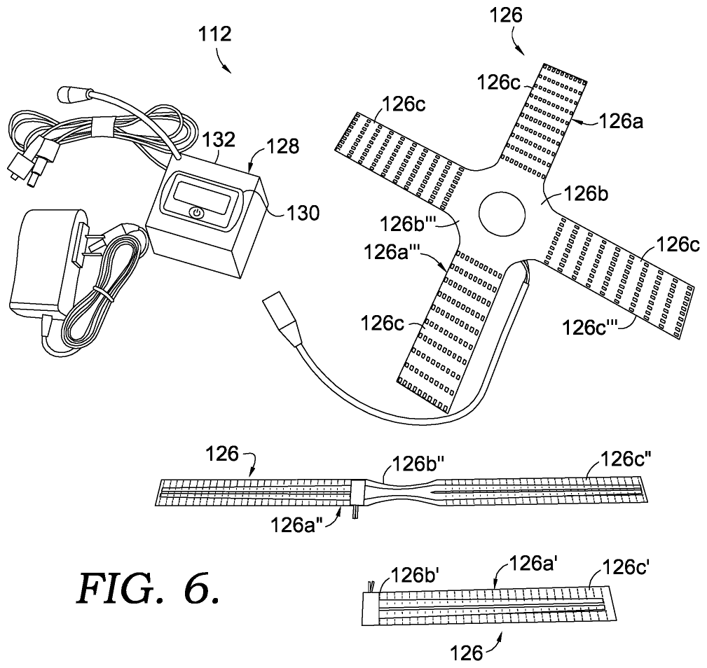

As seen most clearly in FIG. 6, an exemplary heating element 126 within heating apparatus 112 may be a flexible heat strip 126a, although it should be noted that other shapes/sizes of heating elements may be used. In accordance with an aspect of the present invention, flexible heat strip 126a may comprise a base member 126b and one or more heating units 126c extending outwardly from base member 126b. For example, and without limitation thereto, flexible heat strip 126a′ may have one heating unit 126c′ and base 126b′; flexible heat strip 126a″ may have two heating units 126c″ and base 126b″, or flexible heat strip 126a′″ may have four heating units 126c′″ and base 126b′″). Heating units 126c may then extend along at least a portion of sidewall 116 of orthotic appliance 110. Flexible heat strip 126a may include a flexible heat strip, such as but not limited to a silicon rubber heater, a polymer thick film heater or a thin film heater. The flexible heat strips may be configured to emit heat when supplied with an electrical current from a power supply 128. In one aspect of the present invention, power supply 128 may be a battery.

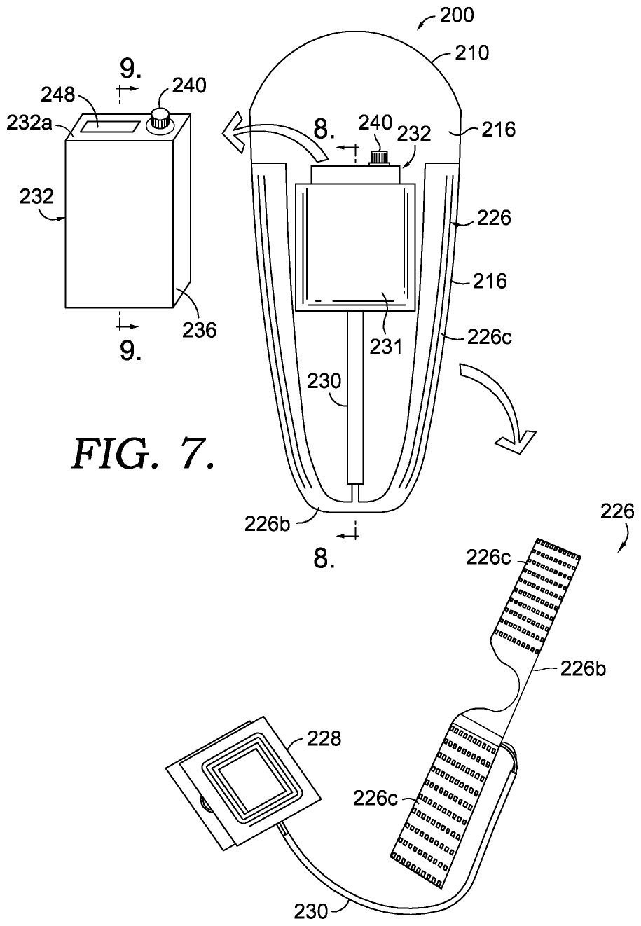

A temperature control unit 130 may regulate the current provided to heating units 126c so as to prevent excessive heating of orthotic appliance 110 and possible injury to the user's limb 120 within interior area 118. Alternatively or additionally, the heat strip may be a positive temperature coefficient (PTC) heater capable of self-modifying so as to output a consistent, user-selected maximum temperature. To prevent overheating and potentially burning a user, the maximum thermal output of heating units 126c may be selected when manufacturing PTC flexible heating units 126c. Heating element 126 may thus have a maximum temperature output selected such that a surface temperature of the orthotic appliance 110 does not exceed about 80 degrees Celsius. Battery 128 and temperature control unit 130 may be packaged within a single housing 132 or separately. Orthotic appliance 110 may further include a mounting feature 134, such as but not limited to a bracket, sleeve or VELCRO strap (not shown, but see, e.g., FIGS. 7-9), configured to couple battery 128 and/or housing 132 to sidewall 116 of orthotic appliance 110.

In accordance with another aspect of the present invention, and with specific reference to FIGS. 7-9, an alternative exemplary embodiment of a temperature adjustment element 226 may be substantially identical to temperature adjustment element 126 described above, but be configured for wireless power. As a result, orthotic appliance 110 may be modified as an orthotic appliance 210 which has been adapted to receive temperature adjustment element 226 to form exemplary orthotic device 200, as will be described in greater detail below.

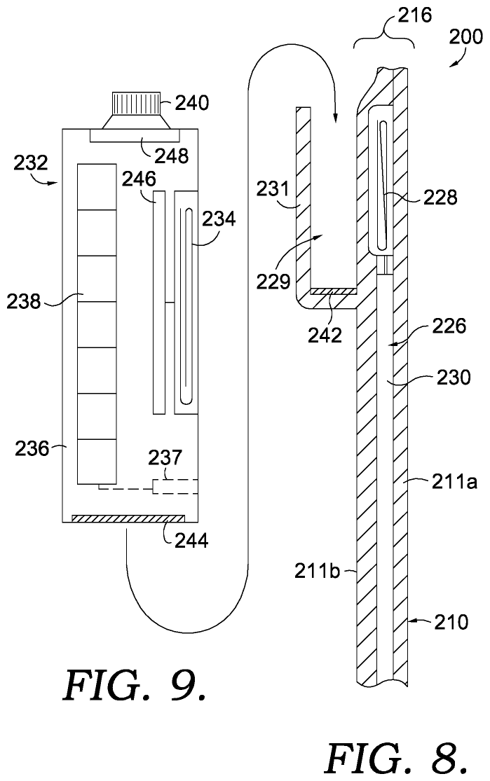

In one aspect of the present invention, heating element 226 may include a wireless charging receiver coil 228 electrically coupled to flexible heat strip 226a, such as via a wired connection 230. Similar to flexible heat strip 126a, flexible heat strip 226a may comprise a base member 226b and one or more heating units 226c. As shown most clearly in FIG. 8, wireless charging receiver coil 228 may be positioned between inner/outer laminate layers 211a/211b of orthotic appliance 210 proximate a pack opening 229 defined by a pack sleeve 231 and outer laminate layer 211b. In one aspect of the present invention, pack sleeve 231 may be integrally formed with sidewall 216. As a result, orthotic appliance 210 may be constructed so as to be waterproof, and more preferably hermetically sealed, such that heating element 226 including wireless charging receiver coil 228 can be used in harsh weather (e.g., rain or snow) without risk of getting wet or suffering an electrical short.

An electric current may be induced within wireless charging receiver coil 228 when a battery pack 232 including a wireless charging transceiver 234 is placed in pack opening 229 in close proximity to wireless charging receiver coil 228, such as via the Qi wireless standard, as will discussed in greater detail below To prevent overheating and potentially burning a user, the maximum thermal output of heating units 226c may be selected when manufacturing PTC flexible heating units 226c.

With continued reference to FIGS. 7-9, battery pack 232 may generally comprise a battery housing 236 which contains one or more batteries 238 arranged therein. Batteries 238 may be electrically coupled to wireless charging transceiver 234 and an ON/OFF switch or button 240. When button 240 is toggled to its ON position, electrical current is supplied to wireless charging transceiver 234 by batteries 238. With wireless charging transceiver 234 properly oriented with wireless charging receiver coil 228, the electrical current passing through wireless charging transceiver 234 may magnetically induce wireless charging receiver coil 228 to produce an electrical current which can then drive thermal output from heating units 226c to warm orthotic appliance 110 and the user's limb received therein. Battery housing 236 may also be waterproof, and more preferably hermetically sealed, such that the battery pack 232 may be used in conjunction with orthotic appliance 110 in harsh weather conditions without risk of electrical shorting or shocking the wearer. Heat may still be provided to the wearer through the magnetically induced current without requiring plugs or other connectors which may be susceptible to water intrusion.

In accordance with an aspect of the present invention, batteries 238 are rechargeable batteries, such as lithium ion batteries, which may be recharged by locating battery pack 232 proximate a wireless charging docking station (not shown). The wireless charging docking station may be a dedicated station or may be any suitable commercially available docking station. The wireless charging docking station may then induce a current in wireless charging transceiver 234 which is used to recharge batteries 238. It should be noted that while battery pack 232 has been shown and described as waterproof and with batteries 238 being recharged through magnetic induction, those skilled in the art would understand that other systems and methods for recharging batteries 238 may be used, such as but not limited to plugging battery pack 232 into a standard wall outlet through a USB or micro-USB adapter port 237.

With reference to FIG. 8, a first magnet 242 of a magnet pair may be mounted on pack sleeve 231 or within sidewall 216 proximate pack opening 229. Battery pack 232 may include the second magnet 244 of the magnet pair such that, when properly seated within pack opening 229, first and second magnet pair 242/244 attract one another so as to removably secure battery pack 232 to orthotic appliance 210 and prevent unwanted dislodging of the battery pack during use. First and second magnet pair 242/244 may also assist in properly aligning wireless charging transceiver 234 with wireless charging receiver coil 228 so as to warm heating units 226c when battery pack 232 is switched ON as described above. Instead of the above-referenced magnet pair, it should be understood that one magnet and a ferromagnetic material may be used to removably secure battery pack 232 in pack opening 229.

In accordance with another aspect of the present invention, battery pack 232 may further include a control board/microcontroller 246 and an indicator 248. Indicator 248 may be located so as to be viewable when battery pack 232 is positioned within pack opening 229, and more preferably is located along a top face 232a of the battery pack 232. In one exemplary embodiment, indicator 248 may comprise a series of light emitting diodes (LEDs) which may represent the charge status of batteries 238. By way of example and without limitation thereto, indicator 248 may include 6 LEDs arranged in a line, where each solid ON indicates ⅙ of the battery charge. Thus, 3 solid ON LEDs and one flashing LED would indicate that the battery charge remaining is between 50-66%, for example. When batteries 238 are being recharged, the number of flashing LEDs may indicate recharge status. As in the above example, 4 flashing LEDs would indicate that the batteries are charging and that the recharging is between 50-66% complete.

In an alternative embodiment, indicator 248 may comprise a display capable of producing alphanumeric characters. By way of example and without limitation thereto, the display may be configured to show a projected battery lifetime, such as by displaying a time expressed as “3 HR 24 MIN” and/or a percentage such as 46% as determined by programmed microcontroller 246 logic. When charging, the display can present a “charging” icon, such as a stylized lightning bolt and/or may display the text “Charging”. In either presentation of battery charging, the display may further present a “percent charged”, such as 56%.

Indicator 248 may further include provisions for presenting the status of the wireless transmission between wireless charging transceiver 234 and wireless charging receiver coil 228, as well as between wireless charging transceiver 234 and the wireless charging docking station. By way of example and without limitation thereto, indicator 248 may include additional LEDs dedicated to indicating transmission. For instance, one or more LEDs may be provided wherein the color status of the LED is indicative of the transmission state. In one example, an LED may be a Blue/Red colored LED whereby when colored Red, there is a fault in the transmission; when colored Blue the transmission is working properly; and when unlit the LED indicates that no receiver has been detected.

The foregoing description of the preferred embodiment of the invention has been presented for the purpose of illustration and description. It is not intended to be exhaustive nor is it intended to limit the invention to the precise form disclosed. It will be apparent to those skilled in the art that the disclosed embodiments may be modified in light of the above teachings. The embodiments described are chosen to provide an illustration of principles of the invention and its practical application to enable thereby one of ordinary skill in the art to utilize the invention in various embodiments and with various modifications as are suited to the particular use contemplated. Therefore, the foregoing description is to be considered exemplary, rather than limiting, and the true scope of the invention is that described in the following claims.

What is claimed is:

1. An orthotic device comprising:
an orthotic appliance formed by at least two laminate layers, wherein the at least two layers includes a first laminate layer and a second laminate layer; and
a heating apparatus including a heating element embedded between the first laminate layer and the second laminate layer.

2. The orthotic device of claim 1 wherein the heating apparatus further includes a power supply operatively coupled to the heating element, wherein the heating element selectively heats the orthotic appliance.

3. The orthotic device of claim 2 wherein the power supply is a battery.

4. The orthotic device of claim 3 wherein the battery is configured to be worn on or proximate to the orthotic appliance.

5. The orthotic device of claim 3 wherein the heating element comprises a flexible heat strip.

6. The orthotic device of claim 5 wherein the flexible heat strip is coupled to the battery via a wired connection.

7. The orthotic device of claim 6 wherein at least one wire passes through an outer surface of the orthotic appliance, wherein the at least one wire is connected between the power supply and the heating element.

8. The orthotic device of claim 2 wherein the heating element includes a wireless receiving coil, wherein the power supply includes a wireless charging transceiver, and wherein when power is supplied to the wireless charging transceiver, a current is induced within the wireless receiving coil to supply power to the heating element.

9. The orthotic device of claim 1 wherein a temperature output of the heating element is selectively adjustable.

10. The orthotic device of claim 1 wherein the heating element has a maximum temperature output selected such that a surface temperature of the orthotic appliance does not exceed about 80 degrees Celsius.

11. A method of selectively heating an orthotic device being worn on at least a portion of a body, the method comprising:
a) placing the orthotic device on the at least a portion of the body, wherein the orthotic device comprises an orthotic appliance formed by at least two laminate layers and a heating apparatus, wherein the at least two laminate layers include a first laminate layer and a second laminate layer, and wherein the heating apparatus includes a heating element embedded between the first laminate layer and the second laminate layer; and
b) selectively powering the heating element to output heat for heating the orthotic device.
